(19) United States
(12) Patent Application Publication
(10) Pub. No.: US 2013/0101984 A1
(43) Pub. Date: Apr. 25, 2013

(54) METHODS OF DETECTING SLEEPINESS

(71) Applicant: THE WASHINGTON UNIVERSITY, St. Louis, MO (US)

(72) Inventors: Paul J. Shaw, St. Louis, MO (US); Matthew Thimgan, St. Louis, MO (US); Laura Gottschalk, St. Louis, MO (US); Stephen P. Duntley, St. Louis, MO (US)

(73) Assignee: THE WASHINGTON UNIVERSITY, St. Louis, MO (US)

(21) Appl. No.: 13/657,340

(22) Filed: Oct. 22, 2012

Related U.S. Application Data
(60) Provisional application No. 61/549,463, filed on Oct. 20, 2011.

(51) Int. Cl. C12Q 1/25; G01N 33/50

(52) U.S. Cl. CPC C12Q 1/25; G01N 33/50
USPC 435/4; 436/129; 436/100

(57) ABSTRACT

The present application is directed to methods of detecting sleepiness.

### CROSS REFERENCE TO RELATED APPLICATIONS

This application claims the benefit of priority of U.S. Provisional Application No. 61/549,463 filed Oct. 20, 2011, which Is hereby incorporated by reference in its entirety.

### GOVERNMENTAL RIGHTS

The invention was made with government support. The government has certain rights in the invention.

### FIELD OF THE INVENTION

The present application is directed to methods of detecting sleepiness.

### BACKGROUND OF THE INVENTION

Forced and self-inflicted sleep loss have reached epidemic proportions in western industrialized populations, costing billions of dollars in lost productivity and creating hazardous conditions on our roadways, in our skies and in our hospitals. Unfortunately, there is no simple quantifiable marker that can detect excessive sleepiness in a subject before the sleepiness results in a serious accident. Current technologies are limited by inter-person variability posing a substantial burden for using current technologies to detect sleepiness in real-world environments (e.g. highway, operating room, cockpit, etc.) or at the point of care (e.g. doctor's office, clinic, etc.). Secondly, existing technologies require sophisticated and expensive equipment, or complicated molecular techniques that are not well suited for obtaining precise measurements, in real-time, in subjects when they are evaluated in noisy and uncontrolled environments (e.g. roadside). Thus the diagnosis of sleep disorders presents a significant challenge for the medical community. Therefore, there is a need for a method to identify sleepiness in individuals to either function safely in today's society or maintain optimal health.

### SUMMARY OF THE INVENTION

One aspect of the invention provides methods for detecting sleepiness in a subject. The method comprises providing a test sample comprising a sample of bodily fluid taken from the subject, determining the concentration of one or more metabolites in the test sample, and comparing the concentration of the metabolite in the sample to a threshold concentration of the metabolite. A concentration of the metabolite that is significantly different in the test sample compared to the threshold concentration of the metabolite indicates sleepiness. Sample metabolites are selected from the group consisting of the metabolites in Table 1 or a combination thereof.

Another aspect of the invention provides methods for detecting sleepiness in a subject using pyrophosphate as a marker for sleepiness. The method comprises providing a test sample comprising a sample of bodily fluid taken from the subject, determining the concentration of pyrophosphate in the test sample, and comparing the concentration of pyrophosphate in the sample to a threshold concentration of pyrophosphate. A concentration of pyrophosphate that is significantly different in the test sample compared to the threshold concentration of pyrophosphate indicates sleepiness.

Another aspect of the invention provides methods for detecting sleepiness in a subject using oxalate as a marker for sleepiness. The method comprises providing a test sample comprising a sample of bodily fluid taken from the subject, determining the concentration of oxalate in the test sample, and comparing the concentration of oxalate in the sample to a threshold concentration of oxalate. A concentration of oxalate that is significantly different in the test sample compared to the threshold concentration of oxalate indicates sleepiness.

Another aspect of the invention encompasses a biomarker for sleepiness in a subject. The biomarker comprises the level of one or more metabolites selected from the group consisting of the metabolites in Table 1 or a combination thereof.

### DETAILED DESCRIPTION OF THE PREFERRED EMBODIMENTS

Using a metabolomics approach, metabolites that are highly correlated with sleepiness have been identified. The levels of the metabolites, as detailed in the Examples, have been shown to be responsive to sleep deprivation in human subjects, making the metabolites suitable biomarkers for identifying and/or predicting dangerous levels of sleepiness. As such, the present invention provides biomarkers and methods that can be used to distinguish between subjects who are awake and excessively sleepy and subjects who are awake and well rested. The method of the invention can thus be used to identify and predict dangerous levels of sleepiness, especially in vulnerable populations, such as pilots, doctors, and commercial truck drivers.

### I. Use of Metabolites to Detect Sleepiness

One aspect of the invention provides a method that generally utilizes the level of one or more metabolites selected from the group consisting of the metabolites in Table 1 to detect sleepiness in a subject. Specifically, as detailed in the Examples, it has been discovered that the level of the metabolites in Table 1 are highly correlated with sleepiness.

TABLE 1
Metabolites

Oxalate
6-aminocaproate
5-aminovalerate
Pyrophosphate
Glycylproline
Glutarate
Urocanate
Acetylphosphate
Glycerol 3-phosphate
Glycerate
2-hydroxyglutarate
Alpha hydroxyisovaerate
Arabinose

The method comprises providing a test sample comprising a sample of bodily fluid taken from the subject. As will be appreciated by a skilled artisan, the method of collecting a test sample from a subject can and will vary depending upon the nature of the test sample. Any of a variety of methods generally known in the art may be utilized to collect a test sample from a subject. Generally speaking, the method preferably maintains the integrity of the metabolite such that it can accurately be quantified in the test sample.

In one embodiment, the test sample is blood. Various methods of collecting blood are known in the art. Some of these methods are directed toward hospital applications (see U.S. Pat. No. 5,286,262), while others are directed more toward personal use at home or elsewhere, i.e., handheld glucose monitoring systems (see U.S. Patent Application 20030143113). Generally, a method of collecting blood comprises accessing the blood using a skin-piercing element and collecting the blood therein into some type of a collection device. Accessing the blood may also involve the use of a fluid pathway, a capillary channel (e.g., a capillary tube), a fluid transfer medium (e.g., a hydrophilic porous material), or some kind of mechanical or vacuum means in conjunction with the skin-piercing element. The steps of accessing the blood sample, collecting the test sample, and measuring the level of amylase in the test sample may be performed as separate steps with separate devices or these steps may be combined and performed using one device.

In an alternative embodiment, the test sample is interstitial fluid. Methods of collecting interstitial fluid are disclosed in U.S. Pat. No. 6,837,988 and U.S. Pat. No. 6,939,312, which are hereby incorporated by reference in their entirety. Generally, a method of collecting interstitial fluid is similar to the method disclosed above with regard to blood. Typically, when accessing interstitial fluid, the skin-piercing element has a reduced penetration depth, as compared to blood; typical skin-piercing elements for accessing interstitial fluid include micro-piercing elements. In certain aspects of the invention, the skin-piercing element is selected from the group consisting of a microneedle, microlancet, or another micro-piercing element. In some particular aspects, a micro-piercing element is present in conjunction with one or more elements selected from the group consisting of a fluid pathway, a fluid medium, a vacuum means, and a mechanical means.

In yet another embodiment, the test sample is pancreatic juice. Several methods of collecting a sample of pancreatic juice are known in the art. For example, a sample of pancreatic juice may be obtained by a conventional duodenal tube after exogenous secretin; the duodenal tube aspirates pancreatic juice from the duodenum. Alternatively, a sample of pancreatic juice may be obtained by endoscopic cannulation.

In a further embodiment, the test sample is urine. A variety of methods of collecting a urine sample are also known in the art. Typically, for example, the subject urinates into a sterilized container, such as a cup.

In a preferred embodiment, the test sample is saliva. Several suitable methods of collecting saliva are known in the art, ranging from expectoration into a container to the use of an absorbent collection device, i.e., an absorbent swab. Various methods and apparatuses for collecting saliva have also been disclosed and by way of non-limiting example include methods disclosed in U.S. Pat. No. 4,589,548, U.S. Pat. No. 4,580,577, U.S. Pat. No. 4,283,498, U.S. Pat. No. 3,518,164, and U.S. Pat. No. 4,768,238, which are hereby by incorporated by reference in their entirety.

The subject may be any mammal known to suffer from sleepiness or used as a disease model for sleepiness. In one embodiment, the subject is a rodent. Examples of rodents include mice, rats, and guinea pigs. In another embodiment, the subject is a primate. In another embodiment, the subject is a primate. Examples of primates include monkeys, apes, and humans. In an exemplary embodiment, the subject is a human. In some embodiments, the subject has no clinical signs of sleepiness. In other embodiments, the subject has mild clinical signs of sleepiness. In yet other embodiments, the subject may be at risk for sleepiness. In still other embodiments, the subject has been diagnosed with sleepiness.

The method typically comprises determining the concentration of one or more metabolites from Table 1 in a test sample. In some embodiments, the concentration of 1, 2, 5, 10, or 13 of the metabolites in Table 1 may be measured. In other embodiments, the concentration of 1, 2, 3, 4, 5, 6, 7, 8, 9, 10, 11, 12, or 13 of the metabolites in Table 1 may be measured. In a preferred embodiment, the concentration of one of the metabolites in Table 1 may be measured. In an exemplary alternative of this embodiment, the concentration of oxalate may be measured. In another exemplary alternative of the embodiment, the concentration of pyrophosphate may be measured.

In another preferred embodiment, the concentrations of two of the metabolites in Table 1 may be measured. In an exemplary alternative of this embodiment, the concentrations of oxalate and pyrophosphate may be measured.

As will be appreciated by a skilled artisan, the method of determining the concentration of one or more metabolites can and will vary depending upon the nature of the test sample and the metabolite to be measured. Non-limiting examples of methods that may be used to determine metabolite concentration may include LC-MS, HPLC, GC-MS, enzyme-coupled spectrophotometric assays. In some embodiments, the method used for measuring the concentration of the metabolite is a method suitable for multiplex concentration determination.

The method comprises comparing the concentration of a metabolite to a threshold concentration of the metabolite. In one embodiment, the threshold concentration of the metabolite may be the level of the metabolite in a well rested subject. In one alternative of the embodiment, an elevated level of the metabolite in the same subject, compared to the threshold level of the metabolite, indicates sleepiness. In another alternative of the embodiment, a reduced level of the metabolite in the same subject, compared to the threshold level of the metabolite, indicates sleepiness.

In another embodiment, a threshold level of the metabolite may refer to the average level of the metabolite in a particular population, measured when the population is well-rested. In one alternative of the embodiment, an elevated level of the metabolite in an individual of the population, compared to the threshold level of the metabolite for the population when well-rested, indicates sleepiness. In another alternative of the embodiment, a reduced level of the metabolite in an individual of the population, compared to the threshold level of the metabolite for the population when well-rested, indicates sleepiness.

Methods of measuring whether a subject is well-rested are known in the art, and include, but are not limited to, the Stanford sleepiness scale.

### II. Types of Sleepiness

A variety of types of sleepiness or sleep related disorders may be detected according to the practice of the invention. The methods of the present invention, for example, may be used to distinguish between subjects who are awake and excessively sleepy and subjects who are awake and well rested. The term “sleepiness” is used broadly herein and encompasses sleep deprivation, coma, sleep fragmentation and disorders or conditions generally known in the art or otherwise described herein. Additionally, sleepiness may be used to refer to sleep drive. Numerous sleep disorders have been identified and may be diagnosed utilizing the methods of the invention. Sleep disorders can be grouped into four general categories: dyssomnias, parasomnias, sleep disorders associated with medical/psychiatric disorders, and proposed sleep disorders.

In one embodiment, the method may be used to detect or treat a subject having a dyssomnia. Dyssomnias typically can be divided into three groups: intrinsic sleep disorders, extrinsic sleep disorders, and circadian rhythm sleep disorders. It is contemplated that the present invention may be utilized to diagnose and treat any of the aforementioned sleep disorders. Intrinsic sleep disorders include psychophysiological insomnia, sleep state misperception, idiopathic insomnia, narcolepsy, recurrent hypersomnia, idiopathic hypersomnia, posttraumatic hypersomnia, obstructive sleep apnea syndrome, central sleep apnea syndrome, central alveolar hypoventilation syndrome, periodic limb movement disorder, rhythmic movement disorder, and restless legs syndrome. Extrinsic sleep disorders include inadequate sleep hygiene, environmental sleep disorder, altitude insomnia, adjustment sleep disorder, insufficient sleep syndrome, limit-setting sleep disorder, sleep-onset association disorder, food allergy insomnia, nocturnal eating (drinking) syndrome, hypnotic-dependent sleep disorder, stimulant-dependent sleep disorder, alcohol-dependent sleep disorder, and toxin-induced sleep disorder. Circadian rhythm sleep disorders include time zone (jet lag) syndrome, shift work sleep disorder, irregular sleep-wake pattern, delayed sleep phase syndrome, advanced sleep phase syndrome, and non 24-hour sleep-wake disorder.

In an alternative embodiment, the method may be used to detect or treat a subject having parasomnia. Parasomnias typically can be divided into three groups: arousal disorders, parasomnias usually associated with REM sleep, and other parasomnias. The present invention contemplates diagnosis and treatment of any of the aforementioned disorders. Arousal disorders include confusional arousals, sleepwalking, sleep talking, and sleep (or night) terrors. Parasomnias usually associated with REM sleep may include nightmares, sleep paralysis, impaired sleep-related penile erections, sleep-related painful erections, REM sleep-related sinus arrest, and REM sleep behavior disorder. Other parasomnias may include sleep bruxism, sleep enuresis, sleep-related abnormal swallowing syndrome, nocturnal paroxysmal dystonia, sudden unexplained nocturnal death syndrome, primary snoring, infant sleep apnea, congenital central hypoventilation syndrome, sudden infant death syndrome, and benign neonatal sleep myoclonus.

The method may also be used to diagnose and treat a subject having a sleep disorder associated with a medical or psychiatric condition. Sleep disorders associated with medical/psychiatric disorders can be grouped into three categories: those associated with mental disorders, those associated with neurological disorders, and those associated with other medical disorders. The present invention contemplates diagnosis and treatment of any of the aforementioned disorders. Sleep disorders associated with mental disorders may include psychoses, i.e., schizophrenia, mood disorders, i.e., bipolar disorder, depression, anxiety disorders, panic disorders, and alcoholism. Sleep disorders associated with neurological disorders may include cerebral degenerative disorders, dementia, parkinsonism, fatal familial insomnia, sleep-related epilepsy, electrical status epilepticus of sleep, and sleep-related headaches. Sleep disorders associated with other medical disorders may include sleeping sickness, nocturnal cardiac ischaemia, chronic obstructive pulmonary disease, sleep-related asthma, sleep-related gastroesophageal reflux, peptic ulcer disease, and fibrositis syndrome.

A variety of other sleep disorders may be diagnosed and treated according to the practice of the invention. These sleep disorders may include, for example, short sleeper, long sleeper, subwakefulness syndrome, fragmentary myoclonus, sleep hyperhidrosis, menstrual-associated sleep disorder, pregnancy-associated sleep disorder, terrifying hypnogogic hallucinations, sleep-related neurogenic tachypnea, sleep-related larnyngospasm, and sleep choking syndrome.

The method may also be used to identify pharmaceuticals that may induce sleepiness. In one embodiment, the method may be used to identify pharmaceuticals with sleepiness as an adverse side effect. In another embodiment, the method may be used to identify pharmaceuticals with sleepiness as a desired primary effect.

A variety of subjects may be diagnosed or treated according to the practice of the invention. The subject may be any human or animal subject who has sleepiness or a sleep disorder or sleep condition or who is at risk for developing any of the aforementioned indications. The subject may be a domestic livestock species, a laboratory animal species, a zoo animal or a companion animal. In one embodiment, the subject is a mammal. In an exemplary embodiment, the mammal is a human being. In certain aspects of the invention, the subject has been diagnosed with a sleeping disorder. Numerous sleeping disorders have been identified, as discussed in detail above. In some embodiments of the invention, the subject is at risk for sleepiness. Those at risk for sleepiness may include individuals with undiagnosed or untreated sleep disorders, i.e., untreated obstructive sleep apnea, business travelers, shift workers, individuals who work long hours, commercial drivers, i.e., long-haul drivers, young people—especially males under age 26, parents of young children, police officers, pilots, health professionals, i.e., physicians, military professionals, individuals being treated with certain sedating medications, i.e., anti-depressants, and individuals who have consumed alcohol or certain illegal drugs.

### III. Kits for Detecting Sleepiness

Another aspect of the invention encompasses kits for detecting sleepiness in a subject. A variety of kits having different components are contemplated by the current invention. Generally speaking, the kit will include a means for detecting in a subject the level of one or more metabolites consisting of the biomarkers in Table 1. In another embodiment, the kit will include means for collecting a test sample, means for measuring the level of one or more metabolite in Table 1 in the test sample, and instructions for use of the kit contents. In some embodiments, the kit comprises a means for measuring the level of oxalate. In other embodiments, the kit comprises a means for measuring the level of pyrophosphate. In yet other embodiments, the kit comprises a means for measuring the levels of oxalate and pyrophosphate.

### EXAMPLES

The following Examples illustrate various iterations of the invention.

### Example 1

### Identification of Sleep Deprivation Metabolites

The Metabolon biochemical profiling platform was employed to identify metabolites in saliva taken from sleep deprived human subjects. A total of 14 subjects were sleep deprived at the Washington University Sleep Laboratory and samples were collected on site. The samples from the 14 sleep deprived subjects were analyzed by Metabolon using mass spectrophotometric techniques.

Thirteen metabolites that are robustly sensitive to sleep loss were identified from the sleep deprived human subjects. These are 1) Oxalate (ethanedioate), 2) 6-aminocaproate, 3) 5-aminovalerate, 4) pyrophosphate (PPi), 5) glycylproline, 6) glutarate (pentanedioate), 7) urocanate, 8) acetylphosphate, 9) glycerol 3-phosphate (G3P), 10) glycerate, 11) 2-hydroxyglutarate, 12) alpha-hydroxyisovalerate, and 13) arabinose.

### Example 2

### Colorimetric Measurement of Metabolite Levels

Since mass spectrophotometric methods to quantify metabolites are not suitable for real-world detection of sleepiness, the samples from Example 1 were subsequently examined using enzymatic colorimetric assays for subsets of metabolites.

### Example 3

### Oxalate and PPi Metabolite Levels in Sleep Deprived Individuals

It was confirmed that 30 hours of sleep loss resulted in a significant reduction in salivary levels of oxalate and PPi. In addition, it was independently confirmed that both oxalate and PPi levels were reduced in independent samples collected from subjects after 24 and 27 hours of wakefulness, respectively. Thus, oxalate and PPi levels are significantly and reliably reduced after waking and these changes are observed in each individual over repeated assessments. Subsequently, an additional 30 individuals were evaluated. These subjects were kept awake for 24 and 48 hours on two separate occasions. Once again, separate analysis of oxalate and PPi levels were significantly reduced in saliva samples taken from subjects that were not allowed to sleep.

Importantly, the data suggest that there is a threshold for these metabolites that seems to define individuals that are sleepy without the necessity to know an individual's non-sleep deprived oxalate levels. In all, 74 independent determinations of metabolite levels were made in 44 subjects. The data showed that the oxalate and PPi levels are a sensitive and reliable measurement of sleep loss and sleepiness.

### Example 4

### Oxalate Levels in Short and Normal Sleepers

A total of 57 subjects from the general population were examined and sleep objectively was evaluated using actigraphy. A significant Pearson correlation was found between total sleep time and oxalate levels (p<0.05). Thus, oxalate is able to distinguish between short and normal sleepers in a real-world setting.

What is claimed is:

1. A method for detecting sleepiness in a subject, the method comprising:
(a) providing a test sample comprising a sample of bodily fluid taken from the subject;
(b) determining the concentration of one or more metabolites in the test sample from (a), wherein the sample metabolites are selected from the group consisting of the metabolites in Table 1 or a combination thereof;
(c) comparing the concentration of the metabolite in (b) to a threshold concentration of the metabolite;
wherein a concentration of the metabolite that is significantly different in the test sample compared to the threshold concentration of the metabolite indicates sleepiness.

2. The method of claim 1, wherein the test sample is selected from the group consisting of a blood sample, an interstitial fluid sample, a saliva sample, a urine sample, and a pancreatic sample.

3. The method of claim 1, wherein the test sample is a saliva sample.

4. The method of claim 1, wherein an elevated level of the metabolite compared to a threshold concentration of the metabolite indicates sleepiness.

5. The method of claim 1, wherein a reduced level of the metabolite compared to a threshold concentration of the metabolite indicates sleepiness.

6. The method of claim 1, wherein the metabolite is oxalate.

7. The method of claim 1, wherein the metabolite is pyrophosphate.

8. The method of claim 1, wherein sleepiness is a condition selected from the group consisting of sleep deprivation, coma, dyssomnias, parasomnias, and sleep disorders.

9. A method for detecting sleepiness in a subject, the method comprising:
(a) providing a test sample comprising a sample of bodily fluid taken from the subject;
(b) determining the concentration of pyrophosphate in the test sample from (a);
(c) comparing the concentration of pyrophosphate in (b) to a threshold concentration of pyrophosphate;
wherein a concentration of pyrophosphate that is significantly different in the test sample compared to a threshold concentration of pyrophosphate indicates sleepiness.

10. The method of claim 9, wherein the test sample is selected from the group consisting of a blood sample, an interstitial fluid sample, a saliva sample, a urine sample, and a pancreatic sample.

11. The method of claim 9, wherein a reduced level of pyrophosphate in the test sample compared to the threshold concentration of pyrophosphate indicates sleepiness.

12. The method of claim 9, wherein the concentration of oxalate in the test sample from (a) is determined.

13. The method of claim 12, wherein a reduced level of pyrophosphate compared to a threshold concentration of pyrophosphate and a reduced level of oxalate compared to a threshold concentration of pyrophosphate and oxalate indicates sleepiness.

14. The method of claim 9, wherein sleepiness is a condition selected from the group consisting of sleep deprivation, coma, dyssomnias, parasomnias, and sleep disorders.

15. A method for detecting sleepiness in a subject, the method comprising:
(a) providing a test sample comprising a sample of bodily fluid taken from the subject;
(b) determining the concentration of oxalate in the test sample from (a);
(c) comparing the concentration of oxalate in (b) to a threshold concentration of oxalate;
wherein a concentration of oxalate that is significantly different in the test sample compared to a threshold concentration of oxalate indicates sleepiness.

16. The method of claim 15, wherein the test sample is selected from the group consisting of a blood sample, an interstitial fluid sample, a saliva sample, a urine sample, and a pancreatic sample.

17. The method of claim 15, wherein a reduced level of oxalate in the test sample compared to the threshold concentration of oxalate indicates sleepiness.

18. The method of claim 15, wherein the concentration of pyrophosphate in the test sample from (a) is determined.

19. The method of claim 18, wherein a reduced level of oxalate compared to a threshold concentration of oxalate, and a reduced level of pyrophosphate compared to a threshold concentration of oxalate and pyrophosphate indicates sleepiness.

20. The method of claim 15, wherein sleepiness is a condition selected from the group consisting of sleep deprivation, coma, dyssomnias, parasomnias, and sleep disorders.
